(19) United States
(12) Patent Application Publication
(10) Pub. No.: US 2009/0081135 A1
(43) Pub. Date: Mar. 26, 2009

(54) Iodine-based oral antibacterial treatment methodology

(76) Inventor: Jeffrey D. Glen, Bala Cynwyd, PA (US)

Correspondence Address: Kenneth P. Glynn, 24 Mine Street, Flemington, NJ, 08822, US

(21) Appl. No.: 11/903,583

(22) Filed: Sep. 24, 2007

(51) Int. Cl. A61K 8/20; A61Q 11/00

(52) U.S. Cl. 424/51

(57) ABSTRACT

An iodine-based oral antibacterial treatment methodology involves applying an effective amount of an iodine-based oral antibacterial treatment to the inside of a human or animal mouth in the gum area between the gum and a tooth wall. The delivered treatment includes available iodine, including molecular iodine, and an iodine free radical initiator selected from the group consisting of chloride ions, bromide ions and mixtures thereof. Animals include pets, such as dogs and cats, as well as farm animals having teeth, e.g., horses, cows, goats and the like, and zoo animals having teeth, such as chimps, baboons, tigers and the like.

### BACKGROUND OF INVENTION

a. Field of Invention

The invention relates generally to iodine-based oral antibacterial treatment methodologies to reduce or eliminate gum diseased areas of humans and of animals, such as pets.

b. Description of Related Art

The following patents are representative of a login system:

U.S. Pat. No. 5,916,581 to Chris Foret et al., describes a stable, aqueous, iodine-based germicidal compositions which include respective amounts of available iodine, non-ionic surfactant (e.g., polyoxyethylene, polyoxypropylene block copolymers) and an iodine-solubilizing halide ion selected from the group consisting of Cl⁻ and Br⁻ and mixtures thereof. Use of Cl⁻ and/or Br⁻ substantially reduces or completely eliminates the need for iodide ion in the compositions for maintaining available iodine in solutions.

U.S. Pat. No. 5,230,895 to Ronald F. Czarnecki et al., describes an in vivo method for treating a subject for periodontal disease by placing a mixture of glyceride composition and a therapeutic agent in the perodontal pocket of subject such that the therapeutic agent is released in a sustained manner. The glyceride composition is selected such that the mixture of the glyceride composition and the therapeutic agent is capable of forming a gel in the environment of the periodontal pocket. Also described is a sustained release delivery system comprising the mixture of the glyceride composition and therapeutic agent.

U.S. Pat. No. 5,002,763 to Robert B. Login et al describes a water soluble complex of polyvinylpyrrolidone, hydrogen chloride and iodine, and process for making the same.

U.S. Pat. No. 5,366,733 to Nancy S. Brizzolara et al., describes oral compositions for the local administration of a therapeutic agent to a periodontal pocket of a patient for alleviating dental disease comprising a plurality of dry, discrete microparticles each of which comprise an effective amount of at least one therapeutic agent dispersed in a matrix comprising a biocompatible and biodegradable polymer. Apparatus and methods are also provided for the dispensing of the dry microparticles to the periodontal pocket whereby they become tacky and adhere to the involved tissue so as to induce long term therapeutic benefits.

U.S. Pat. No. 5,529,770 to Michael D. McKinzie et al., describes improved, highly viscous film-forming protective germicidal formulations are provided which include water, a germicidal agent (preferably complexed iodine) together with an organic thickener, a skin conditioning agent and a minor amount of polyvinyl pyrrolidone. Preferably, the formulations include iodine complexed with a Poloxamer and PVP, in combination with a xanthan gum and glycerin. The formulations have a viscosity of from about 50-5000 cP and form a flexible residual film when applied to skin. The invention is particularly useful in the preparation of bovine teat dips.

Notwithstanding the prior art, the present invention is neither taught nor rendered obvious thereby.

### SUMMARY OF INVENTION

The present invention relates to an iodine-based oral antibacterial treatment methodology that involves applying an effective amount of an iodine-based oral antibacterial treatment to the inside of a human or animal mouth in the gum area, i. e., at the sulcus or otherwise particularly between the gum and a tooth wall. Animals include pets, such as dogs and cats, as well as farm animals having teeth, e.g., horses, cows, goats and the like, and zoo animals having teeth, such as chimps, baboons, tigers and the like.

The delivered treatment of the present invention includes available iodine, including molecular iodine, and an iodine free radical initiator selected from the group consisting of chloride ions, bromide ions and mixtures thereof. The mixture, referred to herein as a treatment, may be in a format selected from the group consisting of powder, aqueous solution, gel, paste and microencapsulation. Other available formats could be used, e.g., a suspension, a non-aqueous solution, a dissolvable film, etc., without exceeding the scope of the present invention.

In some preferred embodiments of the present invention iodine-based oral antibacterial treatment methodology, the methodology involves applying an effective amount of an iodine-based oral antibacterial treatment into the sulcus of the gum area of a human or animal mouth, the treatment containing: (a.) about 3% to about 30% by weight a water soluble non-ionic surfactant; (b.) about 1% to about 12% by weight of available iodine, including molecular iodine; (c.) about 0.1% to about 50% by weight of a halide ion selected from the group consisting of chloride ions, bromide ions and mixtures thereof; (d.) balance by weight being an aqueous carrier selected from the group consisting of water and aqueous solutions containing inert, non-interfering constituents.

In some preferred embodiments of the present invention iodine-based oral antibacterial treatment methodology, the surfactant is selected from those having the formula:

R1-(CHR2-CHR2-O)ₙ—H

wherein R1 is the residue of an organic compound containing an active hydrogen or hydroxide and R2 is hydrogen or a C₁ to C₄ alkyl group and n is an integer from 3 to 212 ethoxylated fatty alcohols and fatty acids and mixtures thereof.

In some preferred embodiments of the present invention iodine-based oral antibacterial treatment methodology, the surfactant is an amount of about 5% to about 25%.

In some preferred embodiments of the present invention iodine-based oral antibacterial treatment methodology, the halide ion is in an amount of about 0.25% to about 25%.

In some preferred embodiments of the present invention iodine-based oral antibacterial treatment methodology, the halide ion is a bromide ion. In some of these preferred embodiments of the present invention iodine-based oral antibacterial treatment methodology, the bromide ion is selected from the group consisting of sodium bromide and hydrobromic acid.

In some preferred embodiments of the present invention iodine-based oral antibacterial treatment methodology, the halide ion is a chloride ion selected from the group consisting of sodium chloride and hydrochloric acid.

In some preferred embodiments of the present invention iodine-based oral antibacterial treatment methodology, the treatment is delivered to a human gum area in an amount of about 0.1 grams to about 2 grams.

In some preferred embodiments of the present invention iodine-based oral antibacterial treatment methodology, the treatment is encapsulated in dissolvable, consumable microcapsules.

In some preferred embodiments of the present invention iodine-based oral antibacterial treatment methodology, the non-ionic surfactant is selected from the group consisting of polyethoxylated polyoxypropylene block copolymers, alkylphenol ethoxylates having 4 to 12 alkyl groups, ethoxylated fatty alcohols and fatty acids and mixtures thereof.

In other preferred embodiments, the present invention iodine-based oral antibacterial treatment methodology, includes applying an effective amount of an iodine-based oral antibacterial treatment by placement between a gum area and a tooth wall of a human mouth or an animal mouth, the treatment including: (a.) about 3% to about 30% by weight a water soluble non-ionic surfactant; (b.) about 1% to about 12% by weight of available iodine, including molecular iodine; (c.) about 0.1% to about 50% by weight of a halide ion selected from the group consisting of chloride ions, bromide ions and mixtures thereof; (d.) balance by weight being an aqueous carrier selected from the group consisting of water and aqueous solutions containing inert, non-interfering constituents.

In some preferred embodiments of the above present invention iodine-based oral antibacterial treatment methodology, the surfactant is selected from those having the formula:

R1-(CHR2-CHR2-O)ₙ—H

wherein R1 is the residue of an organic compound containing an active hydrogen or hydroxide and R2 is hydrogen or a C₁ to C₄ alkyl group and n is an integer from 3 to 212, ethoxylated fatty alcohols and fatty acids and mixtures thereof.

In some preferred embodiments of the above present invention iodine-based oral antibacterial treatment methodology, the surfactant is an amount of about 5% to about 25%.

In some preferred embodiments of the above present invention iodine-based oral antibacterial treatment methodology, the halide ion is in an amount of about 0.25% to about 25%.

In some preferred embodiments of the above present invention iodine-based oral antibacterial treatment methodology. The iodine-based oral antibacterial treatment methodology of claim 12 wherein the halide ion is a bromide ion selected from the group consisting of sodium bromide and hydrobromic acid.

In some preferred embodiments of the above present invention iodine-based oral antibacterial treatment methodology, the halide ion is a chloride ion selected from the group consisting of sodium chloride and hydrochloric acid.

In some preferred embodiments of the above present invention iodine-based oral antibacterial treatment methodology. The iodine-based oral antibacterial treatment methodology of claim 12 wherein about 0.1 grams to about 2 grams of the treatment is delivered.

In some preferred embodiments of the above present invention iodine-based oral antibacterial treatment methodology, the treatment is encapsulated in dissolvable, consumable microcapsules.

In some preferred embodiments of the above present invention iodine-based oral antibacterial treatment methodology, the non-ionic surfactant is selected from the group consisting of polyethoxylated polyoxypropylene block copolymers, alkylphenol ethoxylates having 4 to 12 alkyl groups, ethoxylated fatty alcohols and fatty acids and mixtures thereof.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

The accompanying drawings, which are included to provide a further understanding of the invention and are incorporated in and constitute a part of this specification, illustrate preferred embodiments of the invention and together with the detail description serve to explain the principles of the invention. In the drawings:

FIG. 1 is a diagrammatic view of the methodology for iodine-based oral antibacterial treatment of the gum area of a human or animal according to the present invention;

FIG. 2 is a diagrammatic view of one preferred embodiment of the methodology for iodine-based oral antibacterial treatment of the gum area of a human or animal according to the present invention; and,

FIG. 3 is an enlarged partial front sectional view of a tooth and gum area of a human or animal with a present invention treatment being applied.

### DETAILED DESCRIPTION OF THE EMBODIMENTS

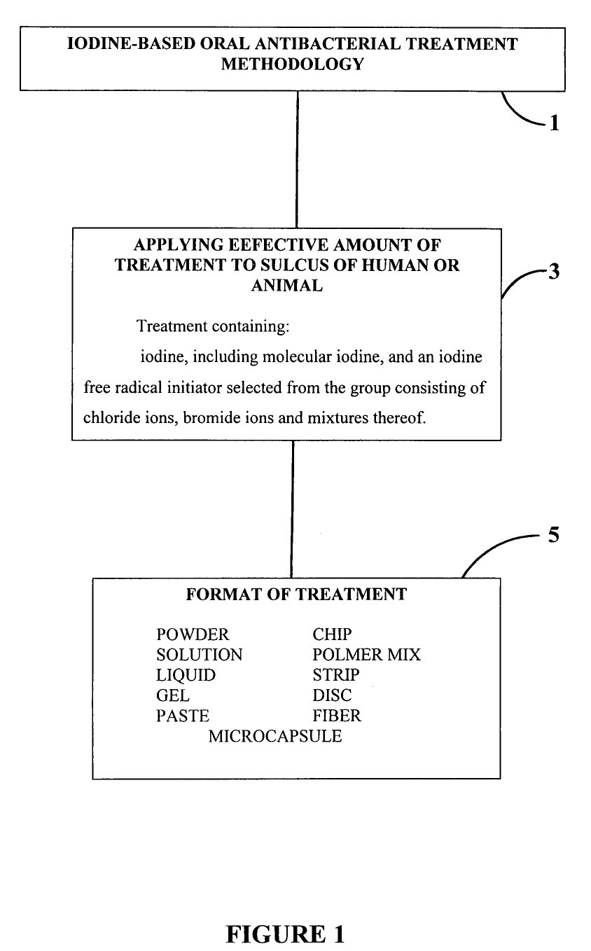

FIG. 1 is a diagrammatic view of the methodology for iodine-based oral antibacterial treatment 1 of the gum area of a human or animal according to the present invention. Block 3 states the treatment methodology and block 5 illustrates some format options.

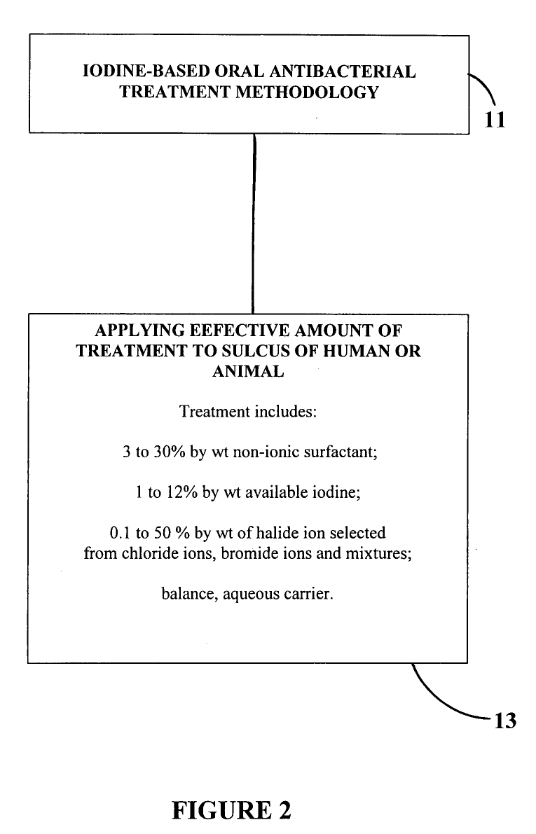

FIG. 2 is a diagrammatic view of one preferred embodiment of the methodology for iodine-based oral antibacterial treatment 11 of the gum area of a human or animal according to the present invention. Block 13 specifies one preferred treatment.

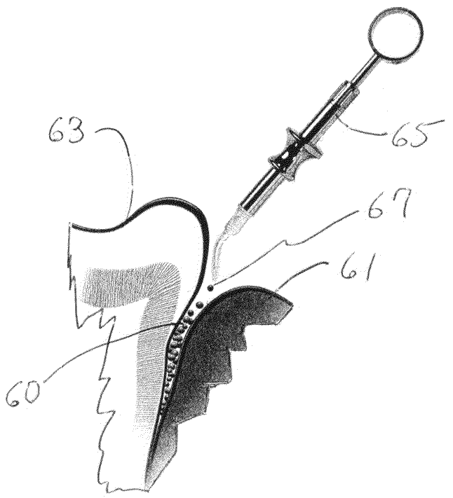

FIG. 3 is an enlarged partial front sectional view of a tooth 63 and gum area 61 of a human or animal with a present invention treatment being applied. A microcapsule delivery syringe 65 is used to deliver microencapsulated treatment 67 to the sulcus 60 between tooth 63 and gum area 61. Edible and consumable encapsulation is a well known art and any safe encapsulation material may be employed, e.g., natural or formulated material such as gelatins and consumable polymers. The treatment may be any formulation within the scope of the invention set forth above in the summary, as well as those set forth below.

The following Examples are representative of some preferred formulations of the present invention treatments:

### EXAMPLE 1

A treatment is prepared using 3.8 g of Pluronic P105, (non-ionic surfactant according to the formula of paragraph [0012] above (Also, see U.S. Pat. No. 5,916,581 issued on Jun. 29, 1999 to Foret et al, incorporated herein in its entirety by reference.) 4.6 g of sodium chloride, 8.3 g of iodine, and 65.3 g of deionized water. These are mixed for four hours at room temperature to yield a concentrated germicidal iodine product. The initial available iodine concentration of this product is 7.7%. After storage for two weeks at 50.degree. C., the available iodine concentration is 7.6%. about 2 grams of this liquid is applied to a sulcus of a human.

### EXAMPLE 2

A concentrated germicidal iodine product was prepared in a glass reaction vessel by combining 19 g of Pluronic P105, 4.8 g of sodium chloride, 0.76 g of sodium iodide and 60 g of deionized water. The mixture was blended until all of the ingredients dissolved completely. Next, 10.5 g of iodine was added to the solution and vigorously mixed. The iodine required approximately 24 hours of mixing at room temperature to dissolve. The initial available iodine concentration of the final product was 9.84% by weight.

### EXAMPLE 3

A concentrated germicidal iodine product was prepared in a glass reaction vessel by combining 13.9 g of Pluronic P105, 9.3 g of sodium bromide, and 61.4 g of deionized water. After dissolution of all the ingredients, 8.4 g of iodine were added. The iodine required approximately 72 hours of mixing at room temperature to dissolve.

### EXAMPLE 4

A concentrate was prepared by combining 27.6 g glycerin, 2.8 g iodine, 4.24 g sodium iodide, 0.1 g acetic acid, 0.2 g sodium acetate, 0.3 g Glucopon 425 (alkyl polyglycoside), and sufficient water to give a total weight of 90 g. This concentrate can be diluted at 1:5 ratio to give a germicidal use solution containing 0.5% iodine and 5% glycerin.

Each of the above treatment formulae may be applied to the sulcus area of a human of animal to treat bacterial diseases and related problems.

Although particular embodiments of the invention have been described in detail herein with reference to the accompanying drawings, it is to be understood that the invention is not limited to those particular embodiments, and that various changes and modifications may be effected therein by one skilled in the art without departing from the scope or spirit of the invention as defined in the appended claims.

What is claimed is:

1. An iodine-based oral antibacterial treatment methodology, comprising:
applying an effective amount of an iodine-based oral antibacterial treatment directly into the sulcus of the gum area of a human mouth or an animal mouth, said treatment containing:
iodine, including molecular iodine, and an iodine free radical initiator selected from the group consisting of chloride ions, bromide ions and mixtures thereof.

2. The iodine-based oral antibacterial treatment of claim 1 wherein said treatment is in a format selected from the group consisting of powder, aqueous solution, gel, paste and microencapsulation.

3. An iodine-based oral antibacterial treatment methodology, comprising:
applying an effective amount of an iodine-based oral antibacterial treatment directly into the sulcus of the gum area of a human mouth or an animal mouth in the gum area, said treatment including:
(a.) about 3% to about 30% by weight a water soluble non-ionic surfactant;
(b.) about 1% to about 12% by weight of available iodine, including molecular iodine;
(c.) about 0.1% to about 50% by weight of a halide ion selected from the group consisting of chloride ions, bromide ions and mixtures thereof;
(d.) balance by weight being an aqueous carrier selected from the group consisting of water and aqueous solutions containing inert, non-interfering constituents.

4. The iodine-based oral antibacterial treatment methodology of claim 3 wherein said surfactant is selected from those having the formula:
R1-(CHR2-CHR2-O)ₙ—H
wherein R1 is the residue of an organic compound containing an active hydrogen or hydroxide and R2 is hydrogen or a C₁ to C₄ alkyl group and n is an integer from 3 to 212, ethoxylated fatty alcohols and fatty acids and mixtures thereof.

5. The iodine-based oral antibacterial treatment methodology of claim 3 wherein said surfactant is an amount of about 5% to about 25%.

6. The iodine-based oral antibacterial treatment methodology of claim 3 wherein said halide ion is in an amount of about 0.25% to about 25%.

7. The iodine-based oral antibacterial treatment methodology of claim 3 wherein said halide ion is a bromide ion selected from the group consisting of sodium bromide and hydrobromic acid.

8. The iodine-based oral antibacterial treatment methodology of claim 3 wherein said halide ion is a chloride ion selected from the group consisting of sodium chloride and hydrochloric acid.

9. The iodine-based oral antibacterial treatment methodology of claim 3 wherein about 0.1 grams to about 2.5 grams of said treatment is delivered to said gum area.

10. The iodine-based oral antibacterial treatment methodology of claim 3 wherein said treatment is selected from the group consisting of liquid, fiber, disc, chip, polymer and encapsulated in dissolvable, consumable microcapsules.

11. The iodine-based oral antibacterial treatment methodology of claim 3 wherein said non-ionic surfactant is selected from the group consisting of polyethoxylated polyoxypropylene block copolymers, alkylphenol ethoxylates having 4 to 12 alkyl groups, ethoxylated fatty alcohols and fatty acids and mixtures thereof.

12. An iodine-based oral antibacterial treatment methodology, comprising:
applying an effective amount of an iodine-based oral antibacterial treatment by placement between a gum area and a tooth wall of a human mouth or an animal mouth, said treatment including:
(a.) about 3% to about 30% by weight a water soluble non-ionic surfactant;
(b.) about 1% to about 12% by weight of available iodine, including molecular iodine;
(c.) about 0.1% to about 50% by weight of a halide ion selected from the group consisting of chloride ions, bromide ions and mixtures thereof;
(d.) balance by weight being an aqueous carrier selected from the group consisting of water and aqueous solutions containing inert, non-interfering constituents.

13. The iodine-based oral antibacterial treatment methodology of claim 12 wherein said surfactant is selected from those having the formula:
R1-(CHR2-CHR2-O)ₙ—H
wherein R1 is the residue of an organic compound containing an active hydrogen or hydroxide and R2 is hydrogen or a C₁ to C₄ alkyl group and n is an integer from 3 to 212, ethoxylated fatty alcohols and fatty acids and mixtures thereof.

14. The iodine-based oral antibacterial treatment methodology of claim 12 wherein said surfactant is an amount of about 5% to about 25%.

15. The iodine-based oral antibacterial treatment methodology of claim 12 wherein said halide ion is in an amount of about 0.25% to about 25%.

16. The iodine-based oral antibacterial treatment methodology of claim 12 wherein said halide ion is a bromide ion selected from the group consisting of sodium bromide and hydrobromic acid.

17. The iodine-based oral antibacterial treatment methodology of claim 12 wherein said halide ion is a chloride ion selected from the group consisting of sodium chloride and hydrochloric acid.

18. The iodine-based oral antibacterial treatment methodology of claim 12 wherein about 0.1 grams to about 2 grams of said treatment is delivered.

19. The iodine-based oral antibacterial treatment methodology of claim 12 wherein said treatment is encapsulated in dissolvable, consumable microcapsules.

20. The iodine-based oral antibacterial treatment methodology of claim 12 wherein said non-ionic surfactant is selected from the group consisting of polyethoxylated poltoxypropylene block copolymers, alkylphenol ethoxylates having 4 to 12 alkyl groups, ethoxylated fatty alcohols and fatty acids and mixtures thereof.
